(19) United States
(12) Patent Application Publication
(10) Pub. No.: US 2002/0099449 A1
(43) Pub. Date: Jul. 25, 2002

(54) Device for use with therapeutic or surgical instruments, implants and equipment therefor

(76) Inventor: Andreas Werner Speitling, Kiel (DE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/005,054

(22) Filed: Dec. 3, 2001

(30) Foreign Application Priority Data
Dec. 6, 2000 (DE) 200 20 649.4

(51) Int. Cl. A61F 2/02

(52) U.S. Cl. 623/23.72; 623/1.42; 424/426

(57) ABSTRACT

An antibacterial device has a surface with a layer that releases ions with an antibacterial effect, e.g. silver ions. The effect of silver is strongly antiseptic even in the bound state, since silver ions contained in the oxide layer of the metal surface exert a blocking effect on the thiol enzymes in the microorganisms. By using a layer that releases silver ions, the risk of bacterial infections can be clearly reduced. Other ions with an antibacterial effect, e.g. copper, can be used by themselves or together with the silver ions. The device may also have a layer having a matrix that is preferably made of plastic. The matrix serves to continuously release silver ions or other ions with an antibacterial effect. With a continuous release of metal ions, a long-lasting antibacterial effect in the tissue is achieved by the surface.

### BACKGROUND OF THE INVENTION

The invention relates to a device for surgical or therapeutic use; it particularly relates to implants, prosthesis and surgical instruments as well as their accessories.

The use of the aforementioned devices sets particular requirements for the materials used. Here, particularly in connection with the use of implants, a requirement that is a primary importance is to achieve good attachment of the surrounding tissue to prosthesis in the implanted state. This is important so that the body's own immune system can be effectively used to defend against bacteria. At the same time, in order to prevent the introduction of bacteria into the body, the device is sterilized before it is used, and freed of germs and bacteria. A disadvantage of this procedure is that a risk of bacterial infection continues to exist for the patient due to handling of the implant device or instrument before and after use.

The invention is based on the task of creating a medical technology for surgical or therapeutic use that reduces the risk of bacterial infections.

### SUMMARY OF THE INVENTION

The device to be kept sterile demonstrates a modified surface that prevents an adhesion of bacteria to the surface and/or possesses an antibacterial effect on the tissue. Both properties can be used jointly, since they are aimed at reducing the number of bacteria on the surface. A reduction of adhesion to the surface has the result that fewer bacteria attach to the device during handling of the device and during implantation, and therefore fewer bacteria are introduced into the human or animal body and multiply there. The bacterial effect is aimed at inactivating or killing bacteria in the tissue that are adhering to or are located in the vicinity of the layer. Both effects can be combined, but each in itself is by itself sufficient to accomplish the task according to the invention.

In a preferred further development of the device, the surface has a layer that releases ions with an antibacterial effect, e.g. silver ions. The effect of silver is strongly antiseptic even in the bound state, since silver ions contained in the oxide layer of the metal surface exert a blocking effect on the thiol enzymes in the microorganisms. Therefore silver ions can have a bacterial effect, for example. By using a layer that releases silver ions, the risk of bacterial infections can be clearly reduced. Other ions with an antibacterial effect, e.g. copper, can be used by themselves or together with the silver ions.

In a preferred further development of the invention, the layer has a matrix that is preferably made of plastic. The matrix serves to continuously release silver ions or other ions with an antibacterial effect. With a continuous release of metal ions, a long-lasting antibacterial effect in the tissue is achieved by the surface.

If the device is made not of metal but rather at least partially of resorbable and non-resorbable plastic, particularly of polylactides (PLA) and poly-L-lactides (PLLA), polyetheretherketone (PEEK), and/or ultra high molecular weight polyethylene UHMWPE, it is preferably to work a substance that releases ions with an antibacterial effect, particularly silver ions, into the plastic mass. Likewise, when using ceramic materials, a substance that releases ions with an antibacterial effect, e.g. silver ions, can be mixed into the ceramic mass. Preferred ceramics are tricalcium phosphate (TCP), hydroxyapatite (HA).

In an alternative further development of the device according to the invention, the surface can be provided with a coating that contains a titanium nitride oxide (TiNOx), titanium niobium ceramic, or titanium zirconium ceramic, and/or an anode oxidation Type II of titanium. These coatings are particularly characterized by their biocompatibility. Furthermore, these coatings possess a surface energy that prevents adhesion of bacteria to the surface, or makes it more difficult. Modified diamond-like carbon (DLC) and/or carbon embedded in steel, even at high concentrations, have proven to be other suitable coatings. Hydroxyapatite coating (HA), calcium phosphate (CaP) coatings, as well as tantalum oxide coating also reduce the adhesion of bacteria, because of their surface properties.

Because of its good biocompatibility and resorbability, it has proven to be particularly advantageous to provide a coating that contains magnesium (Mg).

Preferably, the surface of the device is structured to be smooth, particularly polished and/or ground. This eliminates rough spots that can serve as a point of attachment for bacteria or other contaminants.

In another preferred embodiment of the device according to the invention, the surface has an electrical voltage applied to it, also one with alternating polarity. An electrical voltage has the effect, on certain bacteria, that they find it more difficult to adhere to the surface, and instead are “flushed” off the surface.

In a further development, the surface can have an electrical voltage briefly applied to it. For this purpose, it is possible, for example, that the device is connected with a voltage source for implantation, via a suitable adapter, and that the voltage is applied to the surface in this way.

It is also possible to connect the surface with an alternating voltage source. As an alternative, the surface can also be electrostatically charged.

The task according to the invention is also accomplished by bone cement, particularly polymethylmethacrylate (PMMA) or ceramic cements which may be calcium phosphate cements, or by another substance capable of flow, for medical technology use, which is mixed with a substance that releases ions with an antibacterial effect, particularly silver ions.

### DETAILED DESCRIPTION OF THE PREFERRED EMBODIMENTS

Preferred exemplary embodiments of the device according to the invention will be explained below.

By reducing the number of bacteria that are present on the implant or instrument surface, the risk of a bacterial infection caused by implants and other medical technology devices is reduced. At the same time, if the surrounding tissue attaches well to the implant, the body's own immune system can be effectively used to defend against bacteria.

To reduce the number of bacteria on the implant surface, a physical chemistry surface modification to prevent infection can be used. The approach that is followed in this connection is to prevent the first step of a bacterial infection caused by a medical device (implant, instrument), which is that of adhesion of bacteria to the implant surface. If adhesion of bacteria to the implant surface can be prevented or made more difficult, the risk of a subsequent infection is clearly reduced. This primary adhesion of bacteria can be prevented by means of surface modification. In this connection, the surface energy of the implant surface is changed, by means of suitable surface modification, in such a way that adhesion of bacteria is disadvantageous in terms of energy and biochemistry. In addition, a smooth implant surface is produced, in order to prevent adhesion of bacteria in protected depressions on the surface.

Modification of the surface energy is achieved by means of coating the surface, or by modifying the available regions near the surface, for example by means of diffusion or oxidation processes. A smooth implant surface is achieved by means of grinding and/or polishing that precedes surface modification.

Possible surfaces are:

titanium oxide coating;

modified diamond-like carbon coating;

hydroxyapatite (HA) coating;

calcium phosphate (CaP) coating;

tantalum oxide coating;

titanium niobium ceramic coating;

titanium zirconium ceramic coating;

anode oxidation Type II of titanium;

embedding of carbon in the implant steel; and

magnesium coating.

Such a surface coating can be provided, for example, on implants for osteosynthesis such as plates, screws, pins for external fixators, bone marrow nails. Likewise, such coatings are suitable for implants for joint replacements, such as knee replacements or hip replacements. Such a coating is also advantageous for implants used in the area of the spinal column.

In an alternative embodiment of the device according to the invention, the antibacterial effect of metal ions, particularly silver ions, is utilized. Starting from a defined ion concentration, the bacteria present on the implant surface are killed off, and an infection is therefore prevented. In order to achieve a continuous antibacterial effect on the implant surface, a continuous release of metal ions is preferred.

A substance that releases silver ions or other ions with an antibacterial effect is applied to the implant surface for this purpose. This can be done in a matrix, usually a plastic, or also a ceramic (HA or ICP). The particles located on the surface continuously give off silver ions. Metal implants can be completely or partially provided with a surface coating that releases ions.

In the case of non-metallic implants, for example those made of resorbable or non-resorbable plastics, that is particularly polylactides PLA and poly-L-lactides PLLA, polyetheretherketone (PEEK), ultra high molecular weight polyethylene (UHMWPE), the substance that releases ions can be mixed into the implant material directly, during the production process. This is likewise the case for ceramics, such as tricalcium phosphate (TCP) or hydroxyapatite (HA).

In addition to the implants mentioned above, such substances that release ions can also be mixed into bone cement (polymethylmethacrylate PMMA) and ceramic cement, as well as other substances capable of flow. Likewise, it is possible to mix ion-releasing substances into plastic implants in the orthopedic area.

Another alternative approach takes advantage of the fact that bacteria react to electrical charge. Therefore the adhesion process is influenced by contact of the bacteria with an electrically charged surface. By applying a suitable electrical voltage to the implant, adhesion of bacteria to the implant surface can be prevented, i.e. the surface can be “flushed”.

For this purposes, it is possible to connect a suitable voltage source to the implant after implantation, via a suitable adapter. In this connection, both a voltage that is constantly maintained and short-term application of a voltage are suitable. Also, an alternating voltage can be applied.

The above examples for the production of an infection-resistant, biocompatible surface can also be used for instruments and accessories in the operating room. Maintaining sterile conditions in the operating room is an important prerequisite for avoiding bacterial infections in the hospital. In order to reduce the bacteria count on the instruments and trays used in the operating room, the surfaces of the instruments and trays are modified in such a way that bacteria do not adhere to them, or adhere in reduced number, and/or do not survive on the surface.

It is also possible to use ion-releasing substances for plastic trays, plastic handles, and plastic instruments. Modifications of the surface can also be used in combination with one another.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for surgical or therapeutic use, particularly implants and surgical instruments as well as their accessories, comprising a body with a surface to be kept sterile for use, said surface being modified to have antibacterial effect.

2. The device as set forth in claim 1 wherein the device has an antibacterial layer.

3. The device according to claim 2, wherein the surface has a layer that releases ions with an antibacterial effect.

4. The device as set forth in claim 3 wherein the ions are silver ions.

5. The device according to claim 3, wherein the layer has a matrix, preferably made of plastic, that serves to continuously release ions with an antibacterial effect, particularly silver ions.

6. The device according to claim 1, wherein the body and the surface consisting at least partially of a resorbable and non-resorbable plastic, particularly of polylactides (PLA), poly-L-lactides (PLLA), polyetheretherketone (PEEK). as well as ultra high molecular weight polyethylene (UHMWPE), including a substance that releases ions with an antibacterial effect.

7. The device according to claim 1, wherein the device consists at least partially of ceramic material, particularly of tricalcium phosphate (TCP), hydroxyapatite (HA), which includes a substance that releases ions with an antibacterial effect.

8. The device according to claim 7, wherein the device consists at least partially of ceramic material, particularly of tricalcium phosphate (TCP), hydroxyapatite (HA), which includes a substance that releases ions with an antibacterial effect.

9. The device according to claim 1, wherein the surface is provided with a coating consisting of a member selected from the group consisting of titanium nitride oxide, titanium niobium ceramic, titanium zirconium ceramic, an anode oxidation Type II of titanium and combinations thereof.

10. The device according to claim 1, wherein the surface is provided with a coating that contains modified diamond-like carbon (DLC) and/or carbon embedded in steel.

11. The device according to claim 1, wherein the surface is provided with a coating that contains hydroxyapatite.

12. The device according to claim 1, wherein the surface is provided with a coating that contains calcium phosphate.

13. The device according to claim 1, wherein the surface is provided with a coating that contains tantalum oxide.

14. The device according to claim 1, wherein the surface is provided with a coating that contains magnesium.

15. The device as set forth in claim 1 further comprising a coating consisting of a member selected from the group of hydroxyapatite, calcium phosphate, tantalum oxide, magnesium.

16. The device according to claim 15, wherein the surface is smooth, especially ground and/or polished.

17. The device according to claims 1, wherein the surface has an electrical voltage applied to it.

18. The device according to claim 17, wherein the surface briefly has an electrical voltage applied to it.

19. The device according to claim 17, wherein an adapter is provided to generate an electrical potential at the surface by means of a voltage source, particularly an alternating voltage source.

20. The device according to claim 17, wherein the surface is electrostatically charged.

21. A flowable implantable substance for medical technology use, comprising a substance that releases silver ions with having an antibacterial effect.

22. A method for producing an antibacterial effect on a device for medical use comprising:
incorporating into a surface of the device a member selected from the group consisting of diamond-like carbon, silver ions, copper ions, titanium nitride oxide, titanium niobium ceramic, titanium zirconium, ceramic anode oxidation Type II of titanium, hydroxyapatite, calcium phosphate, tantalum oxide, magnesium and combinations thereof.

23. The method as set forth in claim 22 further comprising applying an electric current to said device.
